(19) United States
(12) Patent Application Publication
(10) Pub. No.: US 2025/0339150 A1
(43) Pub. Date: Nov. 6, 2025

(54) EMBOLIC PROTECTION DEVICES, SYSTEMS, AND METHODS FOR CAROTID PROCEDURES

(71) Applicant: Boston Scientific Scimed, Inc., Maple Grove, MN (US)

(72) Inventors: Aiden Flanagan, Galway (IE); Michael Hughes, Galway (IE); Martin Lawrence Fawdry, Galway (IE); Katie Palmer, Enniskillen (IE); Reyhaneh Shirazi, Galway (IE)

(73) Assignee: Boston Scientific Scimed, Inc., Maple Grove, MN (US)

(21) Appl. No.: 19/195,009

(22) Filed: Apr. 30, 2025

Related U.S. Application Data
(60) Provisional application No. 63/640,987, filed on May 1, 2024.

(51) Int. Cl. A61B 17/12; A61B 17/00; A61B 90/00; A61M 25/10

(52) U.S. Cl. CPC A61B 17/1204; A61B 17/00234; A61B 17/12109; A61B 17/12136; A61B 90/06; A61M 25/10; A61B 2017/00022; A61B 2017/00305; A61B 2017/00398; A61B 2017/00557; A61B 2090/064; A61M 2025/1052; A61M 2205/103; A61M 2205/3327; A61M 2205/3334; A61M 2205/50; A61M 2206/10

(57) ABSTRACT

A reverse flow system for performing vasculature procedures may include a catheter and a flow control system. The catheter may extend from a proximal end to a distal end that is adapted to terminate within a common carotid artery (CCA) and include an occlusion balloon, an inflation lumen in communication with the occlusion balloon, and a working lumen terminating at the distal end of the catheter. The flow control device may be in communication with the working lumen and configured to control a flow rate of fluid through the working lumen and to confirm a full reverse flow of fluid in an internal carotid artery in communication with the CCA and an external carotid artery in communication with the CCA based on a measure related to an amount of force required for the flow control device to achieve the flow rate of fluid through the working lumen.

### CROSS-REFERENCE TO RELATED APPLICATIONS

This application claims the benefit of priority under 35 U.S.C. § 119 of U.S. Provisional Application No. 63/640,987, filed May 1, 2024, the entire disclosure of which is hereby incorporated by reference.

### TECHNICAL FIELD

The disclosure is directed to performing procedures within the carotid artery. More particularly, the disclosure is directed to providing embolic protection during procedures performed in or on carotid arteries.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to several alternative designs, materials and methods of manufacturing medical device structures and assemblies, and the use thereof. An example reverse flow system for performing carotid artery procedures may include a catheter extending from a proximal end to a distal end adapted to terminate within a common carotid artery (CCA), the catheter comprising an occlusion balloon, an inflation lumen in communication with the occlusion balloon, and a working lumen terminating at the distal end of the catheter, a flow control device in communication with the working lumen, and the flow control device may be configured to control a flow rate of fluid through the working lumen and confirm a full reverse flow of fluid in an internal carotid artery (ICA) in communication with the CCA and an external carotid artery (ECA) in communication with the CCA based on a measure related to an amount of force required for the flow control device to achieve the flow rate of the fluid through the working lumen.

Alternatively or additionally to any of the examples discussed herein, the flow control device may be configured to confirm the full reverse flow of fluid in the ICA and the ECA when the measure related to the amount of force required to achieve the flow rate of fluid through the working lumen reaches a threshold value.

Alternatively or additionally to any of the examples discussed herein, the threshold value may be a value of a slope of the measure related to the amount of force required to achieve the flow rate of fluid through the working lumen.

Alternatively or additionally to any of the examples discussed herein, the measure related to the amount of force required to achieve the flow rate of fluid through the working lumen may be a current supplied to a motor of a pump system configured to pump fluid through the working lumen.

Alternatively or additionally to any of the examples discussed herein, the flow control device may include a pump system configured to adjust the flow rate of fluid through the working lumen.

Alternatively or additionally to any of the examples discussed herein, the pump system may include at least one syringe in fluid communication with the working lumen, at least one valve, wherein a valve of the at least one valve may be associated with each of the at least one syringe, at least one motor, wherein a motor of the at least one motor may be in communication with a plunger of each syringe of the at least one syringe to adjust the flow rate of fluid through the working lumen, and at least one sensor configured to sense the measure related to the flow rate of fluid through the working lumen, wherein the measure related to the flow rate of fluid through the working lumen may be a measure related to a force applied by the motor to the plunger to achieve the flow rate of fluid through the working lumen, and the flow control device may further comprise a controller configured to control operation of the at least one motor in response to the measure related to the force applied by the at least one motor to the plunger.

Alternatively or additionally to any of the examples discussed herein, the pump system may include an impeller in-line with the working lumen, at least one motor, wherein the at least one motor may be in communication with the impeller to adjust the flow rate of fluid through the working lumen, and at least one sensor configured to sense the measure related to the flow rate of fluid through the working lumen, wherein the measure related to the flow rate of fluid through the working lumen may be a measure related to a force applied by the at least one motor to the impeller to achieve the flow rate of fluid through the working lumen, and the flow control device may further comprise a controller configured to control operation of the at least one motor in response to the measure related to the force applied by the at least one motor to the impeller.

In another example, a method for establishing reverse flow in an internal carotid artery (ICA) and an external carotid artery (ECA) of a subject may include occluding a common carotid artery (CCA), aspirating blood from the CCA at a flow rate through a working lumen of a catheter, and detecting a total reverse flow of blood from the ICA and the ECA through the working lumen based on a measure related to a force required to achieve aspiring blood from the CCA through the working lumen at the flow rate.

Alternatively or additionally to any of the examples discussed herein, detecting the total reverse flow of blood may include monitoring over time the measure related to the force exerted by a pump system to aspirate blood from the CCA through the working lumen at the flow rate.

Alternatively or additionally to any of the examples discussed herein, detecting the total reverse flow of blood may include identifying a period of time wherein the measure related to the force exerted by the pump system has a first value while aspirating blood from the CCA at the flow rate and identifying the total reverse flow of blood from the ECA and the ICA when the measure related to the force exerted by the pump system increases from the first value to a second value while aspirating blood from the CCA at the flow rate.

Alternatively or additionally to any of the examples discussed herein, the method may further include in response to identifying the total reverse flow of blood, reducing the flow rate at which blood is aspirated from the CCA through the working lumen from a first flow rate to a second flow rate such that the measure related to the force exerted by the pump system remains constant above the second value.

Alternatively or additionally to any of the examples discussed herein, the measure related to the force exerted by the pump system to aspirate blood may be a measure of current applied to a motor of the pump system.

Alternatively or additionally to any of the examples discussed herein, the method may further include in response to detecting the total reverse flow of blood, reducing the flow rate at which blood is aspirated from the CCA through the working lumen from a first flow rate to a second flow rate.

Alternatively or additionally to any of the examples discussed herein, the method may further include maintaining aspiration of blood from the CCA through the working lumen at the second flow rate while performing a procedure on one or more of the CCA, the ECA, and the ICA.

Alternatively or additionally to any of the examples discussed herein, the pump system may include one or more syringes and a motor and monitoring the measure related to the force exerted by the pump system to aspirate blood from the CCA at the flow rate includes monitoring a measure related to a force required to cause a plunger of the one or more syringes to adjust positions while blood aspirates from the CCA at the flow rate.

Alternatively or additionally to any of the examples discussed herein, the method may include if the total reverse flow of blood from the ICA and the ECA through the working lumen is not detected after a period of time, increasing the flow rate at which blood is aspirated from the CCA from a first flow rate to a second flow rate.

Alternatively or additionally to any of the examples discussed herein, the method may include inserting a treatment device through the working lumen to a distal end of the catheter proximate the CCA, and wherein the total reverse flow of blood from the ICA and the ECA through the working lumen may be detected while the treatment device is located at the distal end of the catheter proximate the CCA.

In another example, a non-transitory computer readable medium storing instructions that when executed by one or more processors causes the one or more processors to monitor a measure related to a force exerted by a pump system to aspirate blood at a flow rate from the CCA through a working lumen of a catheter having a distal end positioned at the CCA and detect a total reverse flow of blood from an internal carotid artery (ICA) and an external carotid artery (ECA) through the working lumen based on the measure related to the force exerted by the pump system to aspirate blood at the flow rate from the CCA through the working lumen.

Alternatively or additionally to any of the examples discussed herein, when the processor is caused to detect the total reverse flow of blood, the processor is caused to identify a period of time wherein the measure related to the force exerted by the pump system has about a first value while aspirating blood from the CCA at the flow rate and identify the total reverse flow of blood from the ECA and the ICA when the measure related to the force exerted by the pump system to aspirate blood from the CCA at the flow rate increases from the first value to a second value.

Alternatively or additionally to any of the examples discussed herein, the instructions, when executed by the one or more processors, cause the one or more processors to in response to identifying the total reverse flow of blood, reduce the flow rate at which blood is aspirated from the CCA through the working lumen from a first flow rate to a second flow rate such that the measure related to the force exerted by the pump system remains constant above the second value.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic partial cutaway view of a portion of a human head and neck, illustrating some of the vasculature within the neck;

FIG. 2 is a schematic view of a portion of a human anatomy, including an illustrative flow path formed between an illustrative arterial catheter extending from the femoral artery to the carotid artery and an illustrative venous catheter extending from the femoral vein;

FIG. 3 is a schematic view of a portion of an illustrative reverse flow catheter inserted in a common carotid artery;

FIG. 4 is a schematic view of a portion of the illustrative reverse flow catheter inserted in the common carotid artery depicted in FIG. 3, with a procedure catheter inserted therethrough;

FIG. 5 is a schematic view of a portion of the illustrative reverse flow catheter inserted in the common carotid artery depicted in FIG. 3, with a stent positioned in the interior carotid artery and the common carotid artery;

FIG. 6 is a schematic diagram of an illustrative flow control device;

FIG. 7 is a schematic diagram of an illustrative controller and user interface

FIG. 8 is a schematic diagram of an illustrative flow control device;

FIG. 9 is a schematic diagram of an illustrative chart depicting motor current versus time;

FIG. 10 is a schematic diagram of an illustrative chart depicting motor current versus time;

FIG. 11 is a schematic diagram of an illustrative flow control device;

FIG. 12 is a schematic diagram of an illustrative flow control device coupled with vasculature of a subject;

FIG. 13 is a schematic diagram of an illustrative flow control device coupled with vasculature of a subject; and

FIG. 14 is a schematic diagram of an illustrative method of detecting a total reverse flow in a vessel of a subject.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, it is contemplated that the feature, structure, or characteristic may be applied to other configurations whether or not explicitly described unless clearly stated to the contrary.

A variety of arterial diseases are known. Carotid Artery Disease (CAD) is an example of an arterial disease in which plaque lesions may develop within a patient's carotid artery. Because of the position of the carotid artery, and because the carotid artery normally carries oxygenated blood from the heart towards the brain, it will be appreciated that performing endovascular catheter procedures such as, but not limited to, carotid artery stenting within the carotid artery may cause particles dislodged from the lesion or lesions to flow upwards into the brain during the endovascular catheter procedures. Foreign material entering the brain may have deleterious effects on a patient. While distal protection devices may be used to help capture dislodged particles, such distal protection devices have to cross the lesion in order to reach a position distal of the lesion. The act of advancing and positioning a distal protection device may, in itself, dislodge particles from the lesion.

Proximal protection devices are embolic protection devices that do not have to be advanced across the lesion. In some instances, a proximal protection device such as an arterial catheter may be advanced through a patient's arterial system to a point within the carotid artery. As an example, the proximal protection device may enter the arterial system via the femoral artery, although other access points are contemplated. In some instances, the proximal protection device may reach a point within the common carotid artery, which is proximal of where the common carotid artery bifurcates into the external carotid artery and the internal carotid artery. Inflating an inflatable balloon at a distal end of the arterial catheter can occlude antegrade blood flow through the common carotid artery. By fluidly coupling a proximal end of the arterial catheter with the venous catheter, and because of the pressure differences between the arterial system and the venous system, retrograde blood flow to the venous system may be created from a location in the common carotid artery distal of the inflatable balloon. As a result, any particles or other debris that may be dislodged from the lesion during a process of advancing the arterial catheter through the vasculature distal of the inflation balloon as well as during any interventional process or procedure, such as stenting for example, will flow backwards through the arterial catheter and through the venous catheter and into the venous system in which the particles or other debris will break down.

Different degrees of retrograde blood flow from the common carotid artery through the catheter may be achieved. For example, due to pressure differences in the vessels extending from the common carotid artery and pressure at the common carotid artery, retrograde flow through the catheter may be a partial retrograde flow or a full retrograde flow. To prevent debris from a lesion moving with antegrade flow to the brain, a full retrograde flow should be established. The concepts disclosed herein provide techniques for establishing a full retrograde flow from a location distal of an occluded common carotid artery into the catheter and confirming the full retrograde flow has been established.

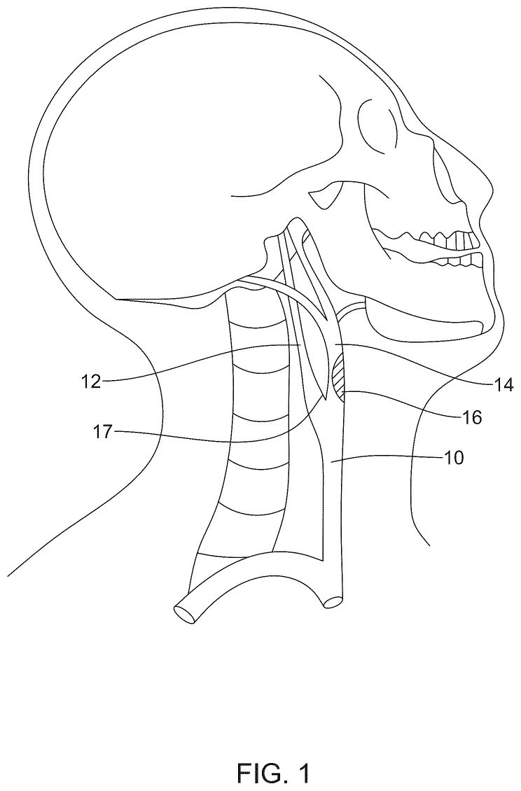

FIG. 1 is a partial cut-away view of the human head and neck, schematically depicting vasculature extending to the human head and neck. FIG. 1 depicts a common carotid artery (CCA) 10, which bifurcates into an external carotid artery (ECA) 12 and an internal carotid artery (ICA) 14. A lesion 16 is schematically shown within the ICA 14, just above a bifurcation point 17. An arterial catheter may be advanced up through the vasculature to a point within the CCA 10 and an inflatable occlusion balloon carried by the arterial catheter may be used to occlude anterograde blood flow through the CCA 10. The CCA 10 may be reached by advancing the arterial catheter through the arterial system to the CCA 10. The arterial system may be accessed via a number of different arteries, but in some instances, the arterial system may be accessed via one of the patient's femoral arteries. In some instances, other arteries providing a shorter path to the CCA 10 may be utilized.

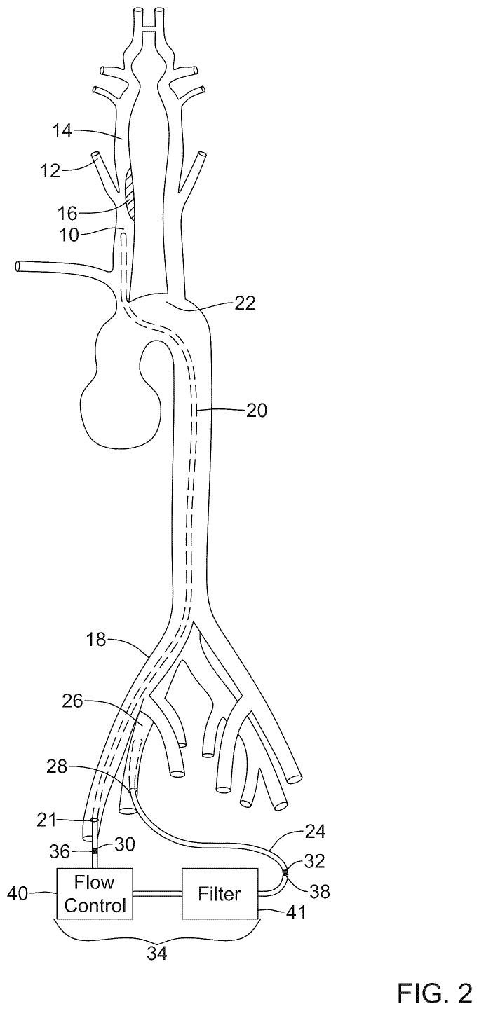

FIG. 2 is a schematic view of a portion of the patient's vasculature providing an illustrative path for advancing an arterial catheter from a femoral artery 18 to the CCA 10. An arterial catheter 20 is schematically seen, passing from an access point 21 within the femoral artery 18, through an aortic arch 22 and into the CCA 10.

Although other suitable techniques may be utilized, a Seldinger technique may be used to create the access point 21 under fluoroscopic guidance. A Seldinger technique may involve introducing a needle into the vasculature, followed by advancing a wire through the needle and into the vessel before the needle is withdrawn. The arterial catheter 20, along with an introducer, may be advanced over the wire and into the femoral artery 18. The arterial catheter 20 may subsequently be advanced through the vasculature to reach the CCA 10, for example.

In some instances, either before or after the arterial catheter 20 has been introduced into the femoral artery 18, a venous catheter 24 may be introduced into the venous system. In some instances, this involves a femoral vein 26, although other access points to the venous system are contemplated. The venous catheter 24 may be introduced into the femoral vein 26 at an access point 28 in a manner similar to that used for introducing the arterial catheter 20 into the femoral artery 18. As an example, a Seldinger technique may be used under fluoroscopic guidance for inserting the venous catheter 24 into the femoral vein 26, but other suitable techniques may be utilized. Although an arterial catheter 20 and a venous catheter 24 are discussed herein as being separate components, a single catheter may utilized for the arterial catheter 20 and the venous catheter 24.

A proximal end 30 of the arterial catheter 20 and a proximal end 32 of the venous catheter 24 may be joined to a fluid path 34. In some examples, the fluid path 34 may represent one or more fittings or connections that allow the proximal end 30 of the arterial catheter 20 and the proximal end 32 of the venous catheter 24 to be fluidly coupled together. The proximal end 30 of the arterial catheter 20 may include a fitting 36 and the proximal end 32 of the venous catheter 24 may include a fitting 38 that permit a direct connection between the proximal end 30 of the arterial catheter 20 and the proximal end 32 of the venous catheter 24 and/or other suitable connections to components of the fluid path 34.

Once the fluid path 34 between the arterial catheter 20 and the venous catheter 24 has been established, the arterial catheter 20 may be advanced further into the femoral artery 18 (or other artery if used) towards the CCA 10. Alternatively or additionally, the arterial catheter 20 may be advanced further toward the CCA 10 prior to and/or while fluidly coupling the arterial catheter 20 with the venous catheter 24.

The fitting 36 and the fitting 38 may each be adapted to be coupled with one or more additional components within the fluid path 34. In one example, the fluid path 34 may include a flow control device 40 configured to couple with the arterial catheter 20 via the fitting 36 and couple with the venous catheter 24 via the fitting 38. Alternatively or additionally, the flow control device 40 may couple with the arterial catheter 20 and/or the venous catheter 24 in one or more other suitable manners.

The flow control device 40 may include one or more components configured to facilitate controlling a flow of fluid through the arterial catheter 20 and/or the venous catheter 24. In some examples, the flow control device 40 may have components including, but not limited to, one or more valves, one or more pumps, one or more sensor, one or more controllers, one or more user interfaces, one or more filters, and/or other suitable components for controlling a flow of fluid through the arterial catheter 20 and/or the venous catheter 24. In some examples, the components of the flow control device 40 may be adjusted by an operator to either permit retrograde blood flow through the fluid path 34, or to prevent retrograde blood flow through the fluid path 34. In some examples, the flow control device 40 may be adapted to be able to adjust the relative retrograde blood flow through the fluid path 34.

As depicted in FIG. 2, the fluid path 34 may include a filter 41, but this is not required. In some examples, the filter 41, when included, may be adapted to screen out any particles over a threshold diameter and/or screen out particles in one or more other suitable manners. In some instances, the filter 41 may be adapted to screen out some particles, while the venous system itself will screen out additional particles.

While the flow control device 40 is shown coupled directly to the arterial catheter 20 and the filter 41 is shown coupled directly to the venous catheter 24, it will be appreciated that this is merely illustrative, as the flow control device 40 and the filter 41 may be connected to each other and/or the arterial catheter 20 and/or the venous catheter 24 in any suitable order or manner. In some example configurations, the fluid path 34 may include the flow control device 40 and may not include the filter 41. In some example configurations, the fluid path 34 may include the filter 41 and may not include the flow control device 40.

A retrograde blood flow (e.g., a reverse flow) may be achieved through the arterial catheter 20 as a result of the arterial catheter 20 being fluidly coupled to a relatively high pressure of the arterial system while the venous catheter 24 is fluidly coupled to a relatively low pressure of the venous system. In some examples, the retrograde blood flow resulting from these pressure differences means that any debris that may be knocked loose or otherwise dislodged while advancing the arterial catheter 20 through the vasculature will be carried through the fluid path 34 into the venous system (e.g., to the extent the debris is not removed when traveling through the fluid path 34). In some examples, at least some debris traveling from the arterial system to the venous system may be captured by the filter 41, when included, including, but not limited to, debris dislodged when placing the arterial catheter 20 in the CCA, debris that may be dislodged while performing various processes (e.g., stenting the lesion 16, etc.), and/or other suitable debris. For example, providing retrograde flow through the arterial catheter 20 may allow for debris that is created or knocked loose during a procedure within the CCA 10, the ECA 12, and/or the ICA 14 to be carried away from the CCA 10, the ECA 12 and/or the ICA 14 in a direction opposite a direction of natural blood flow to the brain through the CCA 10, ECA 12, and the ICA 14.

In some configurations, the arterial catheter 20 may be or may include a balloon catheter. When the arterial catheter 20 is or includes a balloon catheter, the balloon catheter may include at least two lumens. A first lumen (e.g., an inflation lumen) may be in fluid communication with the balloon to facilitate controlling inflation/deflation of the balloon. A second lumen (e.g., a working lumen) of the balloon catheter may be utilized for receiving the retrograde or reverse flow from the CCA 10 and/or for receiving a medical device for performing a procedure distal of the balloon of the balloon catheter In some examples, the balloon catheter may include three lumens, with a first lumen configured as an inflation lumen for the balloon of the balloon catheter, a second lumen configured to receive the retrograde/reverse flow, and a third lumen configured to receive the medical device for performing a procedure distal of the balloon. A distal end of the lumen of the arterial catheter 20 configured to receive the retrograde/reverse flow may be configured to be in fluid communication with the CCA 10 and a proximal end of the lumen configured to receive the retrograde/reverse flow may be in fluid communication with the flow control device 40. Other suitable configurations of the arterial catheter 20 are contemplated.

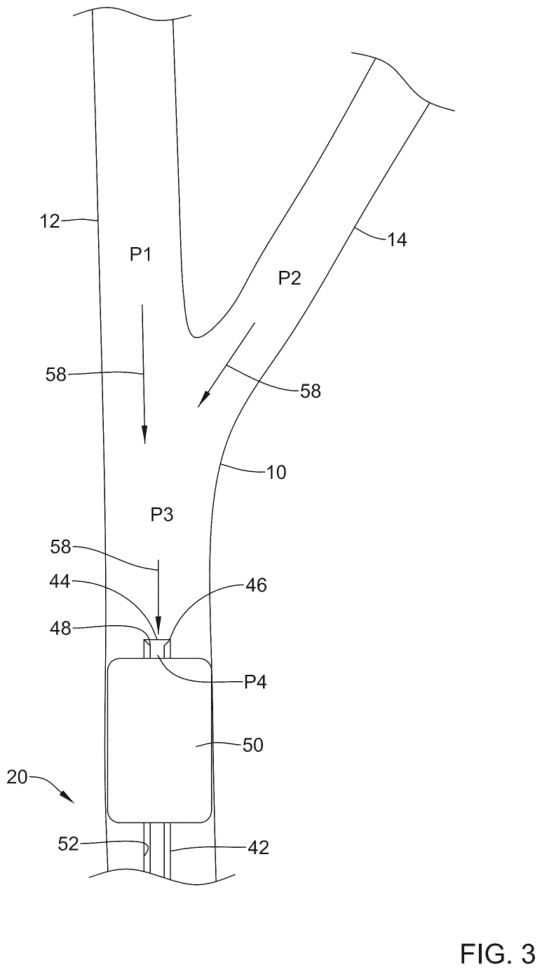

FIGS. 3-5 depict an illustrative procedure for placing a stent 56 in the ICA 14 using a proximal embolic protection device or system configured to ensure and confirm there is full retrograde/reverse flow during a medical procedure (e.g., carotid arterial stenting or other suitable procedure). In one example, after placing the arterial catheter 20 in the CCA 10, a full retrograde/reverse flow of blood from the ECA 12 and the ICA 14 may be initiated, and a stent may be placed in the ICA 14. Alternatively or additionally, other suitable medical procedures may be performed on, at, and/or in the CCA 10, the ECA 12, and/or the ICA 14 after the full retrograde/reverse flow is established and confirmed.

FIG. 3 schematically depicts an illustrative arterial catheter 20 configured as a balloon catheter, with a distal region of the arterial catheter 20 positioned in the CCA 10. The arterial catheter 20 may have a proximal region (e.g., the proximal end 30) fluidly coupled with the venous catheter 24. The arterial catheter 20 may include an elongate shaft 42 that terminates at a distal opening 44 at a distal end 46 of the elongate shaft 42 (e.g., the elongate shaft 42 extends from the proximal end 30 to the distal end 46 of the arterial catheter 20). The distal end 46 of the elongate shaft may be sized and/or otherwise adapted to terminate within the CCA 10. The distal opening 44 may be adapted to permit retrograde/reverse flow into and through a first lumen 48 (e.g., a working lumen) of the elongate shaft 42, where the first lumen 48 may distally terminate at the distal opening 44 and be in fluid communication with the venous catheter 24.

The arterial catheter 20 may include an occlusion balloon 50 that may be inflated in order to occlude the CCA 10 and prevent blood from traveling from the CCA 10 to the ECA 12 and the ICA 14. In some examples, the occlusion balloon 50 may be formed of a compliant silicone and/or may be formed from other suitable types of material. The arterial catheter 20 may include a second lumen 52 (e.g., an inflation lumen) in fluid communication with the occlusion balloon 50 and through which fluid may travel to and from the balloon to inflate and/or deflate the balloon 50. Once the arterial catheter 20 is in place in the CCA 10 and the balloon 50 has been inflated so as to occlude the CCA 10, oxygenated blood may continue to be supplied to the brain through collateral channels from ipsi-lateral vasculature as well as contra-lateral vasculature.

After placing the arterial catheter 20 in the CCA 10, a full retrograde/reverse flow path 58 of fluid from the ECA 12 and the ICA 14 into the distal opening 44 of the arterial catheter 20 may be initiated. In some examples, the full retrograde/reverse flow path 58 of fluid from the ECA 12 and the ICA 14 may be initiated and confirmed using the flow control device 40, as discussed herein or otherwise.

Various pressures are depicted in FIG. 3. For example, the ECA 12 may have a pressure P1, the ICA 14 may have a pressure P2, the CCA 10 may have a pressure P3, and a pressure in or in communication with the first lumen 48 of the arterial catheter 20 may be pressure P4. In operation, when pressure P4 is less than pressure P3, a retrograde/reverse flow from the CCA 10 to the arterial catheter 20 will occur. Then, if pressure P3 is less than pressure P1 in the ECA 12, a retrograde/reverse flow will occur from the ECA 12 to the CCA 10 and the arterial catheter 20. If pressure P3 is less than pressure P2 in the ICA 14, a retrograde/reverse flow will occur from the ICA 14 to the CCA 10 and the arterial catheter 20. If the pressure P3 in the CCA 10 is greater than the pressure P2 in the ICA 14, at least some fluid from the ECA 12 will flow to the ICA 14 and toward the brain of the patient in an undesired manner. Similarly, if the pressure P3 in the CCA 10 is greater than the pressure P1 in the ECA 12, at least some fluid from the ICA 14 will flow to the ECA 12. As a result, full retrograde/reverse flow from the ECA 12 and the ICA 14 may require the pressure P4 in the first lumen 48 of the arterial catheter 20 to be less than the pressure P3 in the CCA 10 and for there to be sufficient flow of fluid from the CCA 10 to the first lumen 48 such that the pressure P3 in the CCA 10 is less than the pressure P1 in the ECA 12 and the pressure P2 in the ICA 14.

In view of the pressure and/or flow requirements needed to ensure there is a full retrograde/reverse flow of fluid from the ECA 12 and the ICA 14, when a retrograde/reverse flow of fluid is observed in the arterial catheter 20 it can only be determined, without further data, that the pressure P4 in the arterial catheter 20 and the pressure P3 in the CCA 10 are less than at least one of the pressure P1 in the ECA 12 and pressure P2 in the ICA 14. That is, it cannot be determined that the pressures P3 and P4 are less than both of the pressures P1 and P2 without sensing pressures in the arterial catheter 20, the CCA 10, the ECA 12, and the ICA 14 and/or receiving other data. Thus, it cannot be conclusively established that there is full retrograde/reverse flow from the ECA 12 and the ICA 14 into the arterial catheter 20, which results in a continuing risk of an embolism occurring. The configurations of the flow control device 40 discussed herein may be configured to confirm there is a full reverse flow of fluid in and/or from the ECA 12 and the ICA 14 into the first lumen 48 of the arterial catheter 20.

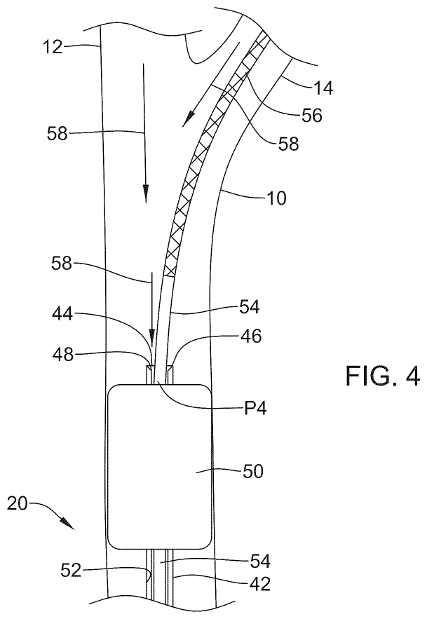

Once a full retrograde/reverse flow is confirmed, a delivery catheter 54 may be advanced out of the distal opening 44 and into the ICA 14, as schematically depicted in FIG. 4. In some examples, a distal region of the delivery catheter 54 may carry one or more expandable or self-expanding stents 56 and/or other suitable medical devices. In addition to or rather than passing the delivery catheter 54 coaxially through the elongate shaft 42 of the arterial catheter 20, the delivery catheter 54 may pass through the balloon 50 at a location adjacent to the elongate shaft 42.

Once the delivery catheter 54 and the stent 56 are positioned in the ICA 14, the stent 56 can be seen as having been deployed. Although not depicted in FIG. 4, the delivery catheter 54 may include a deployment sheath and the deployment sheath may be withdrawn to release the stent for expansion (e.g., self-expansion and/or other suitable expansion) in the ICA 14 and/or the CCA 10. Any debris dislodged as the stent 56 is deployed will follow the path 58 of the retrograde/reverse flow and travel through the distal opening 44 and through the arterial catheter 20 to a filter and/or the venous system.

The full retrograde/reverse flow may be maintained at least until the balloon 50 of the arterial catheter 20 is deflated. In some examples, the flow from the CCA 10 through the arterial catheter 20 may be maintained until the arterial catheter 20 is fully removed from the patient to catch as much debris as possible during movement of the arterial catheter 20 within the patient.

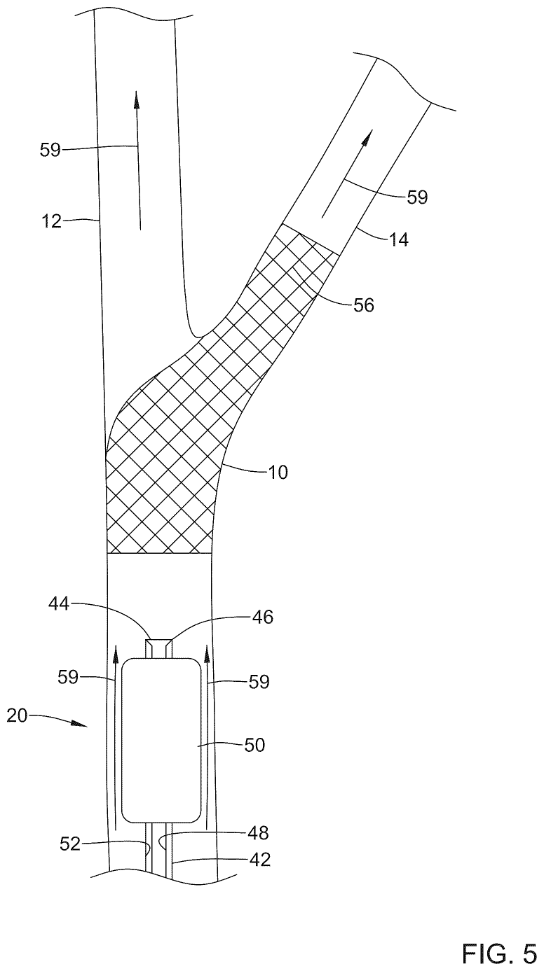

FIG. 5 schematically depicts the stent 56 expanded and positioned in the CCA 10 and the ICA 14. After positioning the stent 56 at a desired location, the delivery catheter 54 may be removed from the CCA 10 and the ICA 14. Subsequently or simultaneously with removing the delivery catheter 54, the balloon 50 may be deflated and the arterial catheter 20 may be withdrawn as well. Once the balloon 50 has been deflated and/or the CCA 10 is no longer fully occluded, aspiration of fluid through the first lumen 48 of the arterial catheter 20 may be stopped or reduced and the arterial catheter 20 may be uncoupled from the venous system to stop the retrograde/reverse flow from the CCA 10, the ECA 12, and/or the ICA 14, which may result in a return of normal or antegrade flow 59 through the CCA 10, the ECA 12, and/or the ICA 14 to the brain of the patient.

Active aspiration using the flow control device 40 may be utilized to achieve full retrograde or reverse flow into the arterial catheter 20 (e.g., via establishing a desired pressure P4) from the CCA 10, the ECA 12, and the ICA 14. The flow control device 40 may be configured to detect and confirm when the full retrograde/reverse flow occurs based on a measure related to a force needed to achieve a desired aspiration flow rate through the arterial catheter 20.

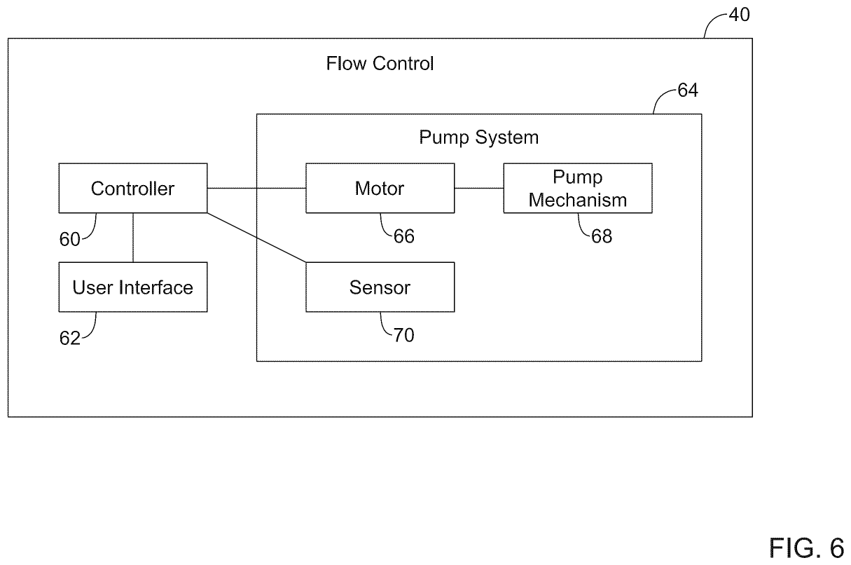

FIG. 6 is a schematic diagram of an illustrative flow control device 40 configured to actively aspirate fluid trough the arterial catheter 20 such that the pressure P4 is low enough to induce a fluid flow from the CCA 10 to the first lumen 48 of the arterial catheter 20 to cause the pressure P3 in the CCA 10 to be less than both of the pressure P1 in the ECA 12 and the pressure P2 in the ICA 14 and attain the desired total retrograde/reverse flow of fluid from the ECA 12 and the ICA 14 into the arterial catheter 20. In some examples, the flow control device 40 may include a controller 60, a user interface 62, a pump system 64, and/or additional or alternative components to actively aspirate fluid from the CCA 10 to ensure and confirm full retrograde/reverse flow of fluid from the ECA 12 and the ICA 14 occurs.

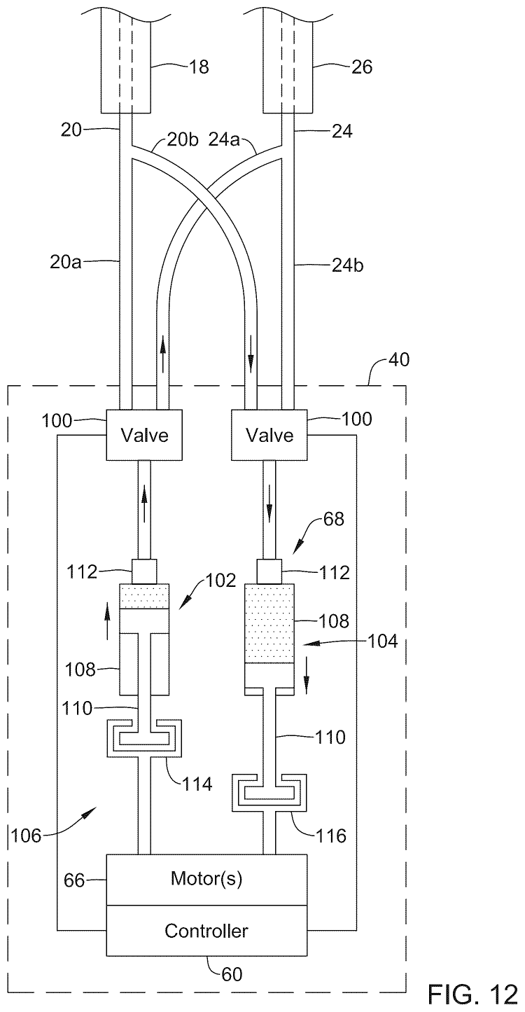

The pump system 64 may include any suitable components. In some examples, the pump system 64 may include one or more motors 66, one or more pump mechanisms 68 configured to pump fluid (e.g., blood) from the arterial system of the patient, through the arterial catheter 20 and the venous catheter 24, and to the venous system of the patient, one or more sensors 70, one or more valves (not shown in FIG. 6, but, for example, see valve(s) 100 in FIG. 12), and/or other suitable components. In one example configuration, the sensor(s) 70 may sense one or more parameters of or related to the motor 66 and/or the pump mechanism 68 related to an amount of force applied to or by the pump mechanism to achieve a desired flow rate of fluid through the arterial catheter 20. The controller 60 may use the measures sensed by the sensor(s) 70 to control operation of the motor 66 and/or the pump mechanism 68 to ensure a full retrograde/reverse flow is achieved and maintained during a procedure.

The one or more motors 66 may be or may include any suitable types of motors. In some examples, the one or more motors 66 may be or may include a direct current (DC) electric motor and/or other suitable types of motors. In one example, the one or more motors 66 may include a brushless DC (BLDC) electric motor, but other suitable motor types are contemplated.

The pump mechanism 68 may be or may include any suitable type of pumping component configured be in fluid communication with the arterial catheter 20 and the venous catheter 24 and to pump fluid in response to activation of the motor 66. For example, the pump mechanism 68 may be or may include one or more pumping components including, but not limited to, one or more syringes, one or more syringe pumps, one or more plungers, one or more impellers, one or more propellers, and/or other suitable pumping components. In one example configuration, the pump mechanism 68 may include dual syringes mechanically coupled to one or more syringe pumps including or in communication with the motor 66 and fluidly coupled to the arterial catheter 20 and the venous catheter 24. In another example, the pump mechanism 68 may include an impeller mechanically, electrically, and/or magnetically coupled with the motor 66 and fluidly coupled with the arterial catheter 20 and the venous catheter 24. Other suitable configurations of the pumping mechanism 68 are contemplated.

The one or more sensors 70 may be or may include any suitable types of sensors configured to be in communication with the controller 60 and to sense a measure related to an amount of force required for the flow control device 40 to achieve a desired flow rate of fluid through the first lumen 48 of the arterial catheter 20. Example suitable types of sensors 70 include, but are not limited to, one or more current sensors configured to sense a measure of current supplied to the motor(s) 66, one or more force sensors configured to sense a force needed to adjust the pump mechanism 68 during aspiration, one or more position sensors for sensing motor or impeller speed, one or more pressure sensors, one or more speed sensors, one or more flow rate sensors, and/or other suitable types of sensors.

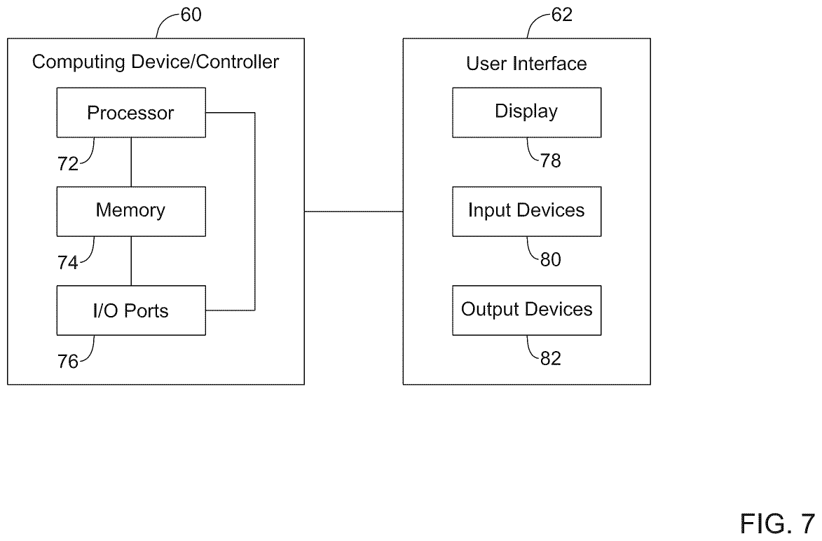

FIG. 7 depicts a schematic diagram of an illustrative configuration of the controller 60 (e.g., computing device) and the user interface 62 of the flow control device 40 and/or of the overall flow system. The controller 60 may be and/or may include any suitable computing device configured to process data of or for the flow control device 40 (e.g., of or from the motor(s) 66, the sensor(s) 70, the pump mechanism 68, etc.) and/or an overall reverse flow system In some cases, one or more components of the flow control device 40 and/or the overall flow system may be incorporated into the controller 60 and/or the user interface 62. Further, one or more components of the flow control device 40 and/or the overall flow system may incorporate one or more computing devices similar to or having components similar to the controller 60 and/or the user interface 62.

The controller 60 may be configured to facilitate operation of the flow control device 40. The controller 60, in some cases, may be configured to control operation of the motor 66, the sensor 70, the user interface 62, and/or the pump mechanism 68 by establishing and/or inputting/outputting control signals to/from components of the motor 66, the sensor 70, the user interface 62, and/or the pump mechanism 68 to control and/or monitor operation of these units and devices.

The controller 60 may communicate with a remote server or other suitable computing device. When the controller 60, or at least a part of the controller 60, is a component separate from a structure of the motor 66, the sensor 70, the user interface 62, and/or the pump mechanism 68, the controller 60 may communicate with electronic components of the flow control device 40 over one or more wired or wireless connections or networks (e.g., LANs and/or WANs).

The controller 60 may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs), software, hardware, firmware, or any combination of these and/or other components. Although the controller 60 may be referred to herein in the singular, the controller 60 may be implemented in multiple instances, distributed across multiple computing devices, instantiated within multiple virtual machines, and/or the like.

The illustrative controller 60 may include, among other suitable components, one or more processors 72, memory 74, and/or one or more input/output (I/O) units 76. Example other suitable components of the controller 60 that are not specifically depicted in FIG. 7 may include, but are not limited to, communication components, a touch screen, selectable buttons, a housing, and/or other suitable components of a controller. As discussed above, one or more components of the controller 60 may be separate from the components of the flow control device 40 and/or incorporated into the components of the flow control device 40.

The controller 60 may include and/or may be in communication with a variety of sub-controllers. Example sub-controllers that may be included in or may be in communication with the controller 60 may include, but are not limited to, a motor sub-controller, a flow rate sub-controller, a pressure sub-controller, pump mechanism sub-controller, and/or other suitable sub-controllers.

The processor 72 of the controller 60 may include a single processor or more than one processor working individually or with one another. The processor 72 may be configured to receive and execute instructions, including instructions that may be loaded into the memory 74 and/or other suitable memory. Example components of the processor 72 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 74 of the controller 60 may include a single memory component or more than one memory component each working individually or with one another. Example types of memory 74 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 74 may be or may include a non-transitory computer readable medium and/or a transitory computer readable medium. The memory 74 may include instructions stored in a transitory and/or non-transitory state on a computer readable medium that may be executable by the processor 72 to cause the processor 72 to perform one or more of the methods and/or techniques described herein.

The I/O unit(s) 76 of the controller 60 may include a single I/O component or more than one I/O component each working individually or with one another. Example I/O units 76 may be or may include any suitable types of communication hardware and/or software including, but not limited to, communication ports configured to communicate with electronic components of the flow control device 10 and/or with other suitable computing devices or systems. Example types of I/O units 76 may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units 76.

The user interface 62 may be configured to communicate with the controller 60 via one or more wired or wireless connections. The user interface 62 may include one or more display devices 78, one or more input devices 80, one or more output devices 82, and/or one or more other suitable features.

The display device(s) 78 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 80 may be and/or may include any suitable components and/or features for receiving user input via the user interface. Example input device(s) 80 include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components), and/or other suitable input devices.

The output device(s) 82 may be and/or may include any suitable components and/or features for providing information and/or data to users and/or other computing components. Example output device(s) 162 include, but are not limited to, displays, speakers, vibration systems, tactile feedback systems, optical outputs, cables, lights, and/or other suitable output devices.

As discussed, the flow control device 40 may be configured to control a flow rate of fluid through from the CCA 10, through the first lumen 48 of the arterial catheter 20 and the venous catheter 24, and to the venous system of the patient (e.g., via a femoral vein or other suitable venous access location). Further, the flow control device 40 may be configured to confirm a full reverse flow of fluid in the CCA 10, the ECA 12, and the ICA 14 into the first lumen 48 of the arterial catheter 20 based on a measure related to an amount of force required for the flow control device 40 to achieve a flow rate of fluid through the first lumen 48 and/or achieve a pump rate (e.g., pump velocity) of the pump mechanism 68. In one example, the measure related to an amount of force required for the flow control device 40 to achieve a desired flow rate of fluid and/or achieve a desired pump rate may be a measure of or related to a current provided to the motor 66 to achieve the desired flow rate and/or pump rate. In another example, the measure related to an amount of force required for the flow control device 40 to achieve a desired flow rate of fluid and/or a desired pump rate may be a measure of or related to a force applied to the pump mechanism 68 to achieve the desired flow rate and/or the desired pump rate. Other suitable measures related to the force required to achieve the desired flow rate and/or the desired pump rate may be utilized.

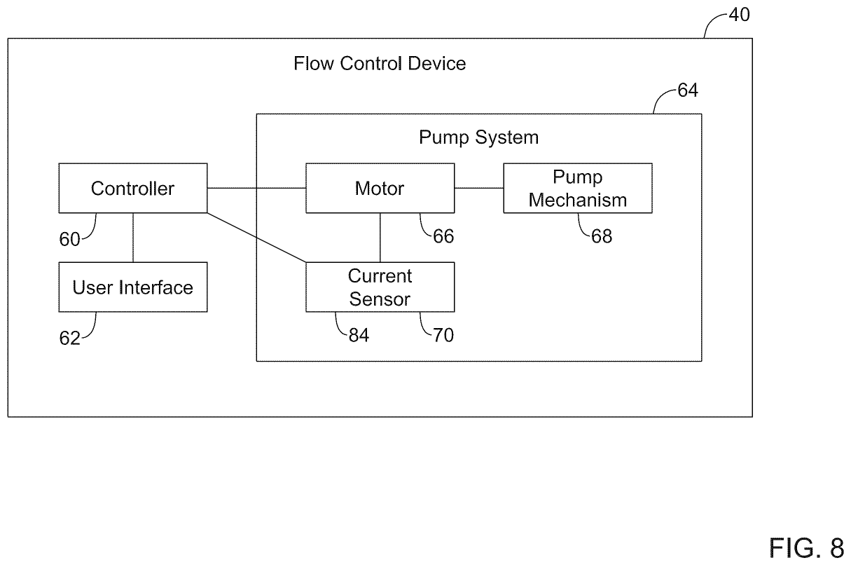

FIG. 8 schematically depicts a diagram of an illustrative flow control device 40 configured to detect and confirm a full reverse flow of fluid in the CCA 10, the ECA 12, and the ICA 14 based on a current (e.g., a motor current) used by the motor 66 to cause the pump mechanism 68 to achieve a desired pump rate and/or actively aspirate fluid from the CCA 10, the ECA 12, and the ICA 14 at a desired flow rate. The desired pump rate and/or the desired flow rate may be any suitable pump rate or flow rate selected to achieve the full retrograde/reverse flow from the CCA 10, the ECA 12, and the ICA 14. As depicted in FIG. 8, the current provided to and/or used by the motor 66 may be sensed by the sensor 70, which may be configured as a current sensor 84, and provided to the controller 60 for monitoring over time and analysis. The current sensor 84 may be part of the motor 66 and/or the controller 60 or separate from the motor 66 and/or the controller 60.

The current may be a measure of the effort the motor exerts to achieve a constant pump rate and/or a constant flow rate and a torque generated by the motor 66 may be directly related to the force applied to the pump mechanism 68 by the motor 66. When the motor receives a control signal to adjust the pump mechanism 68 at a desired speed to achieve a desired flow rate of fluid through the arterial catheter 20, the current used by the motor 66 and sensed by the current sensor 84 is proportional to the force needed to adjust the pump mechanism 68.

As the pump mechanism 68 is adjusted to achieve the desired pump rate and/or flow rate of fluid in the arterial catheter 20, the force needed to adjust the pump mechanism 68 increases to overcome initial inertia, then the force drops and increases gradually until it plateaus. When the desired pump rate and/or the desired flow rate is insufficient to achieve the full retrograde or reverse flow of fluid from the CCA 10, the ECA 12, and the ICA 14, the force plateaus and generally remains steady over time. This plateau of force needed to achieve the desired pump rate and/or desired flow rate is an indication of some fluid flowing through the arterial catheter 20, but not necessarily a full retrograde/reverse flow from the ECA 12 and the ICA 14.

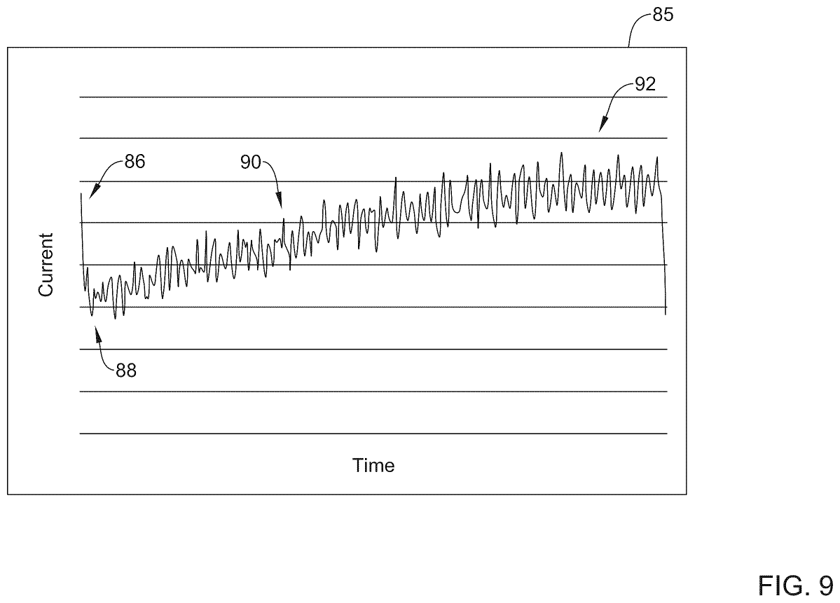

As motor current is proportional to the amount of force needed to achieve a desired pump rate or desired flow rate, motor current may be monitored to determine when the full retrograde/reverse flow occurs. FIG. 9 depicts a schematic chart 85 of sensed motor current versus time in which the sensed motor current (e.g., a measure related to or proportional to the force applied to the pump mechanism 68) has an initial rise 86, then the motor current dips 88, then the motor current steadily rises 90, which is followed by a plateau 92 in motor current, which after a period of time (e.g., any suitable period of time including, but not limited to, less than 1 minute, less than 30 seconds, less than 10 seconds, 5 seconds, and/or other suitable period of time) expires indicates the desired pump rate and/or the desired flow rate of fluid through the arterial catheter 20 is insufficient to achieve full retrograde/reverse flow of fluid. When the desired pump rate and/or the desired flow rate is insufficient to achieve the full retrograde/reverse flow of fluid, the desired pump rate and/or the desired flow rate may be increased, and the motor current or other measure related to force applied to the pump mechanism 68 may be further monitored.

When the desired pump rate and/or the desired flow rate is sufficient to achieve the full retrograde or reverse flow of fluid from the CCA 10, the ECA 12, and the ICA 14, the force starts increasing after a period of time of the plateaued force. The point at which the force required to achieve the desired pump rate and/or the desired flow rate transitions from the plateaued force back to an increasing force is the threshold or transition point at which a full retrograde/reverse flow of fluid from the ECA 12 and the ICA 14 (and thus the CCA 10) occurs.

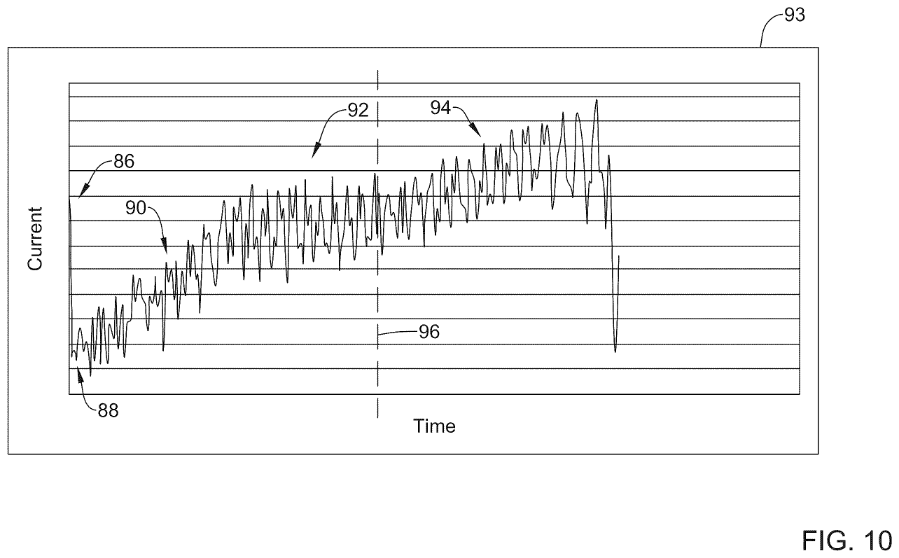

Using motor current measured with the current sensor 84 over time, FIG. 10 depicts a schematic chart 93 of sensed motor current versus time in which the sensed motor current (e.g., an equivalent of the force applied to the pump mechanism 68) has the initial rise 86, then the motor current dips 88, then the motor current steadily rises 90, which is followed by the plateau 92 in motor current, which is then followed by a further steady rise 94 in motor current that is indicative of the full retrograde/reverse flow of fluid from the ECA 12 and the ICA 14. A transition line 96 indicates an illustrative location of the transition from a partial retrograde/reverse flow to a full retrograde/reverse flow of fluid from the ECA 12 and the ICA 14. In some examples, a threshold value at which the flow of fluid through the arterial catheter 20 can be determined to be a full retrograde/reverse flow from the ECA 12 and the ICA 14, along with the CCA 10, may be a value of a change (e.g., a delta) in a slope of the current (or force) at or just after the transition line 96. As such, if the measured current or force has a slope after a plateau slope that results in reaching or going beyond the threshold value for the slope of the current or force, the flow control device 40 may determine a total or full retrograde/reverse flow of fluid in the ECA 12 and the ICA 14 is occurring.

The pressures P1, P2, P3, and P4 discussed above with respect to FIG. 3 may be related to the chart 93 in FIG. 10. In an example, the pressure P4 may be located at a proximal end of the arterial catheter 20 or at or within the pump mechanism 68 (e.g., within a syringe, etc.) fluidly coupled with a proximal end of the arterial catheter 20. When the pressure P3 is located in the CCA 10 and the pressure P4 is at or proximate the proximal end of the arterial catheter 20 and/or in other suitable configurations, there may be a pressure gradient along the first lumen 48 of the arterial catheter 20 between the pressure P3 and the pressure P4. This gradient may be determined by the flow of fluid in the arterial catheter 20, which may be related to a diameter and length of the first lumen 48. When the pump rate of fluid through the first lumen 48 increases, the pressure P4 decreases, the flow rate through the arterial catheter 20 increases, the gradient between the pressure P3 and the pressure P4 increases, and the force required to pump fluid through the arterial catheter 20 increases.

With respect to FIG. 10, the transition line 96 corresponds to when the pressure P3 in the CCA 10 is less than both of the pressure P1 and the pressure P2. At the transition line 96 or just after the transition line 96 may be when the full reverse/retrograde flow into the arterial catheter 20 can be confirmed. For example, the pump mechanism 68 may begin pumping fluid at a pump rate, which may reduce the pressure P4 rapidly and cause a force required to pump fluid at the pump rate or flow rate to increase for a period of time until a flow of fluid through the arterial catheter 20 increases and the pressure P4 and the force required to achieve the pump rate or flow rate plateau (e.g., at the plateau 92 in FIG. 10). If the flow through the arterial catheter 20 is adequate, the pressure P3 in the CCA 10 begins to reduce. In the example, when flow is initiated into the arterial catheter 20, one of but not both of the pressure P1 or the pressure P2 may be less than P3 and therefore flow may be reversed from one of the ECA 12 or the ICA 14 but not the other. Take the example case where P1<P3<P2: As the pressure P3 reduces further, a greater proportion of the flow from the ECA 12 enters the arterial catheter 20 and less proportion of the flow from ECA 12 gets directed to the ICA 14; P3 and P2 continue to reduce however P1 remains relatively constant; this situation occurs in the pump force plateau 92 in FIG. 10. When the pressure P3 becomes less than the pressure P1 and the pressure P2, full reverse/retrograde flow begins. A similar analysis can be made for the example case of P2<P3<P1, with the roles of P1 and P2 in the above example reversed.

Due to limited collateral flow feeding fluid to the ECA 12 and the ICA 14, the pressure P1 and P2 will reduce over time, which may cause the pressure P4 at or proximate the proximal end of the arterial catheter 20 to reduce and the force at the pump mechanism 68 required to maintain the pump rate or the flow rate to increase, which may be represented at the second steady rise 94 in FIG. 10. After a full reverse/retrograde flow is identified, the pump rate or flow rate may be reduced so as to maintain a steady force on the pump mechanism 68 over time.

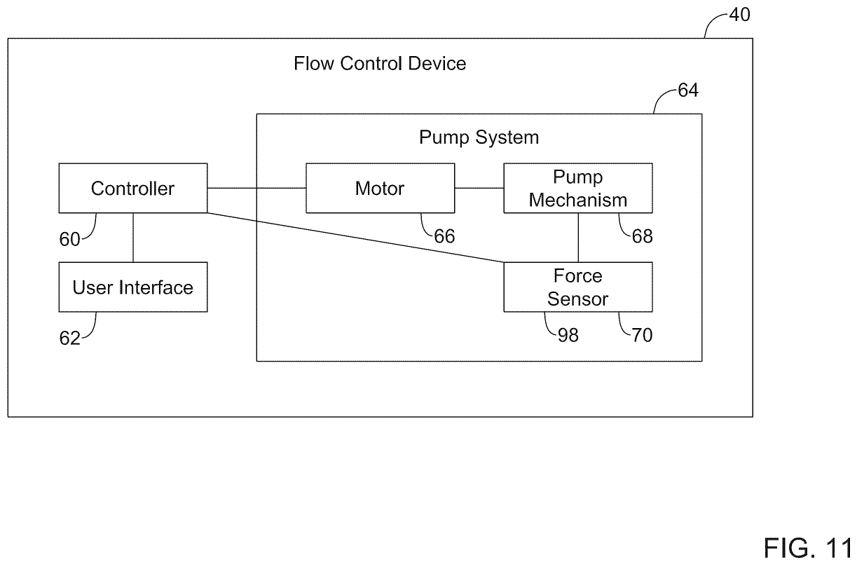

FIG. 11 schematically depicts a diagram of an illustrative flow control device 40 configured to detect and confirm a full reverse flow of fluid in the CCA 10, the ECA 12, and the ICA 14 based on a force applied to the pump mechanism 68 to cause the pump mechanism 68 to reach a desired pump rate and/or to actively aspirate fluid from the CCA 10, the ECA 12, and the ICA 14 at a desired flow rate. The desired pump rate and/or the desired flow rate may be any suitable pump rate or flow rate selected to achieve the full retrograde/reverse flow from the CCA 10, the ECA 12, and the ICA 14.

As depicted in FIG. 11, the force applied to the pump mechanism may be sensed by the sensor 70, which may be configured as a force sensor 98, and provided to the controller 60 for monitoring and analysis. When the pump mechanism 68 includes syringes, the force sensor 98 may be placed between an actuator of a syringe pump and a plunger of the syringe to measure an axial or linear force applied to the plunger. In another example, when the pump mechanism 68 includes an impeller, the force sensor 98 may be configured to sense a rotational force or torque applied to the impeller.

The controller 60 may receive the measures of force and monitor and analyze the sensed measures to determine when a full retrograde/reverse flow of fluid from the ECA 12 and the ICA 14 through the arterial catheter 20 occurs. The controller 60 may look for a same or similar pattern to the patterns in the force measurements as were discussed with respect to FIGS. 9 and 10 for motor current measurements.

FIG. 12 depicts a schematic diagram of an illustrative configuration of the flow control device 40 fluidly coupled with the arterial catheter 20 inserted into the femoral artery 18 of a patient and the venous catheter 24 inserted into the femoral vein 26 of the patient. Although the filter 41 is not depicted in FIG. 12, the filter 41 may be included in the flow path between the arterial system and the venous system and may be upstream or downstream from the flow control device 40. As depicted in FIG. 12, the flow control device 40 may include, among other suitable components, the controller(s) 60 and a pumping system (e.g., the pump system 64 or other suitable pump system) with the one or more motors 66, the pump mechanism(s) 68, a plurality of valves 100, and one or more sensors 70 (not depicted).

Although other suitable pump mechanisms 68 are contemplated, the pump mechanism(s) 68 may include a first syringe 102, a second syringe, and a syringe pump 106. The syringes 102, 104 may each include a syringe body 108, a plunger 110, and a nozzle 112.

The syringe pump 106 may be configured to engage the first syringe 102 and the second syringe 104. The syringe pump 106 may have any suitable configuration for engaging the first syringe 102 and the second syringe 104. In some examples, the syringe pump 106 may include a first actuator 114 configured to engage the plunger 110 of the first syringe 102 and a second actuator 116 configured to engage the plunger 110 of the second syringe 104. The first and second actuators 114, 116 may engage the plungers 110 in any suitable manner including, but not limited to, a keyed fit, a mechanical connection, a magnetic connection, and/or other suitable engagement.

Although the one or more sensors 70 of the flow control device 40 are not depicted in FIG. 12, when the sensors 70 are force sensors the sensors 70 may be positioned between a distal contact portion of the actuators 114, 116 abutting or otherwise proximate a proximal contact portion of the plungers 110 and/or positioned at one or more other suitable locations for measuring force applied to the plungers 110 by the actuators 114, 116. When the sensors 70 are motor current sensors the sensors 70 may be positioned at or proximate the motor 66 and/or positioned at one or more other suitable locations for measuring current supplied to or used by the motor 66. Other configurations of the sensors 70 may be located at the noted locations and/or at other suitable locations.

The first actuator 114 and the second actuator 116 may be actuated in any suitable manner. In some examples, the first actuator 114 and the second actuator 116 may be actuated by the motor(s) 66. The motor(s) 66 may include a single motor that is configured to actuate both of the first actuator 114 and the second actuator 116. Alternatively, the motor(s) 66 may include one or more motors 66 that are configured to actuate the first actuator 114 and one or more motors 66 that are configured to actuate the second actuator 116. Other suitable configurations of the motor(s) 66 relative to the actuators 114, 116 are contemplated.

The valves 100 may be in fluid communication with the arterial catheter 20 and the venous catheter 24. Additionally or alternatively, there may be a valve 100 for each of the syringes 102, 104. In one example configuration, the valve 100 associated with the first syringe 102 may be in fluid communication with a first extension 20a of the arterial catheter 20 and a first extension 24a of the venous catheter 24, while the valve 100 associated with the second syringe 104 may be in fluid communication with a second extension 20b of the arterial catheter 20 and a second extension 24b of the venous catheter 24, as depicted for example in FIG. 12. When so configured, the pumping mechanism 68 may ensure a continuous flow from the arterial catheter 20 to the venous catheter 24 such that the valve 100 associated with a syringe 102, 104 drawing fluid into the syringe body 108 is open to the arterial catheter 20 and the valve 100 associated with the other syringe 102, 104 pushing fluid out of the syringe body 108 may be open to the venous catheter 24 and the positions of the respective valves 100 change with a change in direction of movement of the plungers 110. In some examples, the controller 60 may coordinate movement of the plungers 110 with positions of the valves 100.

The valve(s) 100 may be any suitable type of valve for controlling fluid into and/or out of the pump mechanisms 68. For example, the valves 100 may be, but are not limited to, mechanical valves, electromechanical valves, one-way or check valves, and/or other suitable types of valves.

A position of the valve 100 may be associated with a movement of the actuators 114, 116. In some examples, when the valves 100 are electromechanical valves, the valves 100 may be in communication with the controller 60 to receive control signals. For example, when the second actuator 116 is adjusting or moving to withdraw the plunger 110 associated with the second syringe 104, the valve 100 associated with the second syringe 104 may be controlled to be open to the arterial catheter 20 and closed to the venous catheter 24 and when the first actuator 114 is adjusting to advance the plunger 110 associated with the first syringe 102, the valve 100 associated with the first syringe 102 may be controlled to be open to the venous catheter 24 and closed to the arterial catheter 20. When the valves 100 are one-way or check valves (e.g., mechanical valves) and/or in other suitable configurations (e.g., when the valves 100 may be electromechanical, etc.), each valve 100 may include a first sub-valve associated with the extensions 20a, 20b from the arterial catheter 20 and a second sub-valve associated with the extensions 24a, 24b from the venous catheter 24, where the first sub-valves open and the second sub-valves close (e.g., automatically and/or in response to a control signal) when the plunger 110 withdraws within the syringe body 108 and the first sub-valves close and the second sub-valves open (e.g., automatically and/or in response to a control signal) when the plunger 110 advances within the syringe body 108.

The controller 60 may be configured to communicate with the motor(s) 66 to cause the motor(s) 66 to actuate the actuators 114, 116 such that the pump mechanism 68 achieves a desired pump rate and/or a desired flow rate. In some examples, a desired pump rate and/or a desired flow rate of fluid through the arterial catheter 20 may be set at the flow control device 40 and the controller 60 may output control signals to the motor(s) 66 to achieve the desired pump rate and/or the desired flow rate. Additionally or alternatively, the controller 60 may be in communication with the valve(s) 100 to control a direction of flow to or from the pump mechanism 68.

The controller 60 may receive measures from the sensor(s) 70 and analyze the measures received to determine whether a full retrograde/reverse flow of fluid from the ECA 12 and the ICA 14 has been achieved. When it is determined the full retrograde/reverse flow of fluid has been achieved, the controller 60 may perform a control protocol to maintain the full retrograde/reverse flow of fluid during a procedure in one or more of the CCA 10, the ECA 12, the ICA, and/or other location distal of the occlusion balloon 50. When it is determined the full retrograde/reverse flow of fluid has not been achieved after a predetermined duration of time, the controller 60 may perform a control protocol to adjust (e.g., increase) the pump rate and/or the flow rate of fluid traveling through the arterial catheter 20 in order to achieve the full retrograde/reverse flow of fluid.

In operation, when the controller 60 is coupled with the pump mechanism 68 including the first syringe 102 and the second syringe 104, the controller 60 may control the motor(s) 66 such that one of the syringes 102, 104 is receiving fluid from the arterial catheter 20 and the other of the syringes 102, 104 is outputting fluid to the venous catheter 24 to maintain a continuous flow of fluid from the arterial catheter 20 to the venous catheter 24. As depicted in FIG. 12, the controller 60 may control the first actuator 114 and the valve 100 (optionally) associated with the first syringe 102 to advance the plunger 110 into the syringe body 108 of the first syringe 102 and output fluid to the venous catheter 24. Further the controller 60 may simultaneously control the second actuator 116 and the valve 100 (optionally) associated with the second syringe 104 to withdraw the plunger 110 in the syringe body 108 of the second syringe 104 to receive fluid from the arterial catheter 20. Then, the actuation of the first actuator 114 and the second actuator 116 may be reversed and the process repeated to maintain fluid flow through the catheters 20, 24.

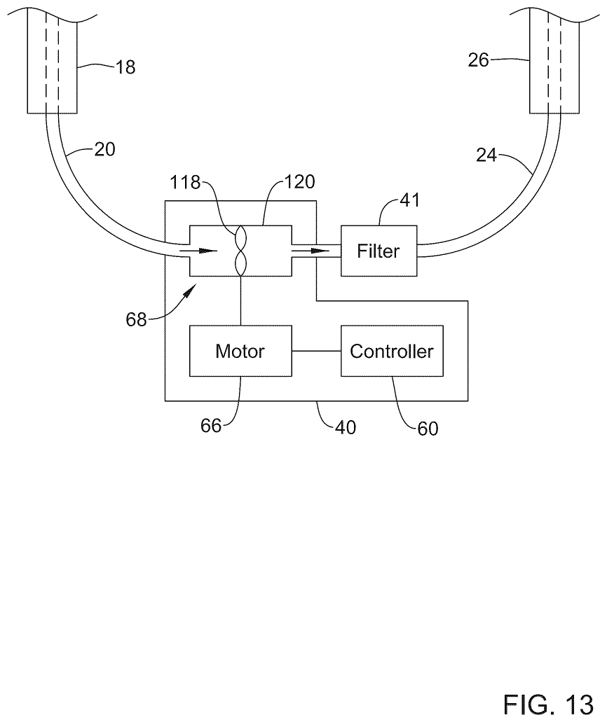

FIG. 13 depicts a schematic diagram of an illustrative configuration of the flow control device 40 and the filter 41 fluidly coupled with the arterial catheter 20 inserted into the femoral artery 18 of patient and the venous catheter 24 inserted into the femoral vein 26 of the patient. Although the filter 41 is depicted downstream of the flow control device 40 in FIG. 13, the filter 41 may be positioned upstream of the flow control device 40, as desired.

The flow control device 40 may include, among other suitable components, the controller(s) 60, the one or more motors 66, the pump mechanism(s) 68, and one or more sensors 70 (not depicted in FIG. 13). Although other suitable pump mechanisms 68 are contemplated, the pump mechanism(s) 68 may include an impeller assembly having one or more impellers 118 and an impeller housing 120. In some examples, the impeller 118 may be located within the impeller housing 120 fluidly coupled with the arterial catheter 20 and the venous catheter 24 and in-line with at least the first lumen 48 (not identified in FIG. 13) of the arterial catheter 20, but other suitable configurations are contemplated.

Although the sensors 70 are omitted from the flow control device 40 in FIG. 13, one or more sensors 70 may be configured to sense a force that a drive shaft from the motor 66 applies to the impeller 118 (e.g., directly, via an impeller shaft, etc.), an amount of current provided to or used by the motor 66, and/or other suitable measure related to a force required to drive the impeller 118 to achieve a desired flow rate of fluid into the first lumen 48 of the arterial catheter 20. When the sensors 70 are included, the controller 60 may analyze sensed measures received from the sensors 70 and output control signals to the motor 66 to cause the motor 66 to actuate the impeller 118. In some examples, the controller 60 may receive a desired flow rate of fluid from a user, output control signals to the motor 66 to achieve the desired pump rate and/or the desired flow rate, receive, from the sensors 70, sensed measures related to the amount of force needed to achieve the desired pump rate and/or the desired flow rate, analyze the sensed measures received from the sensors 70, determine whether a full retrograde/reverse flow of fluid in the ECA 12 and the ICA 14 (and thus, the CCA 10) has been achieved, and then output controls signals to further control the active aspiration caused by the impeller 118 to achieve and/or maintain full retrograde/revers flow of fluid.

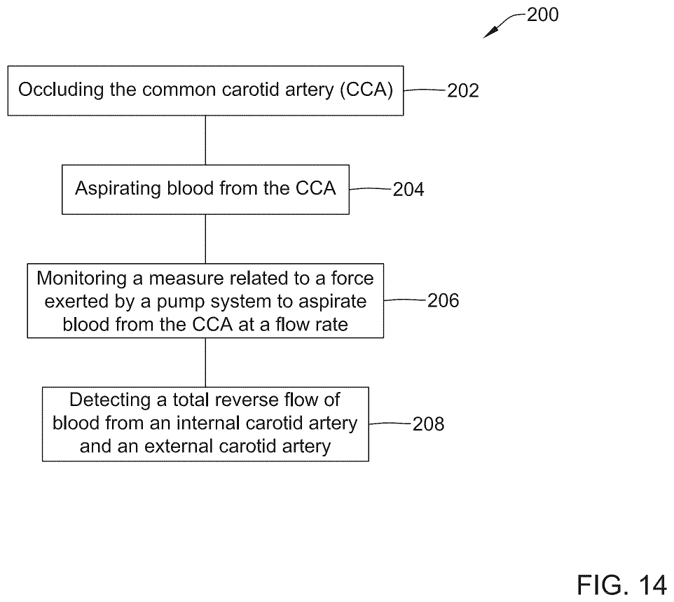

FIG. 14 depicts a schematic diagram of an illustrative method 200 of or for establishing a full retrograde/reverse flow of fluid in the external carotid artery (ECA) and the internal carotid artery (ICA). Although various steps are depicted in FIG. 14, additional and/or alternative steps may be utilized in the method 200 as discussed herein and/or otherwise.

The method 200 may include occluding 202 the common carotid artery (CCA). As discussed herein, the CCA may be occluded in any suitable manner including, but not limited to, by providing an occlusion balloon in the CCA, by clamping the CCA, and/or by occluding the CCA in one or more other suitable manners. In one example, the CCA may be occluded by inserting a balloon catheter (e.g., an arterial catheter) through a femoral artery and into the CCA. Once the balloon catheter is at the CCA, an occlusion balloon of the balloon catheter may be inflated to occlude the CCA at a location proximal of a bifurcation point where the CCA separates into the ECA and the ICA. The balloon catheter may have a distal opening distal of a location of the balloon to facilitate aspirating fluid from the CCA, the ECA, and the ICA. When the CCA is occluded in a manner other than with a balloon catheter, other suitable arterial catheters having a distal opening positioned in the CCA, ECA, and/or the ICA distal of the occlusion at the CCA may be utilized. In some cases, a nominal aspiration through the distal opening of the balloon catheter or other suitable arterial catheter may be activated as the balloon catheter and/or other suitable arterial catheter is advanced through the vasculature to the CCA.

Once the CCA is occluded and the distal opening of the balloon catheter and/or other suitable arterial catheter is located distal of the occlusion in the CCA, fluid (e.g., blood, particles in blood, etc.) may be aspirated 204 (e.g., actively aspirated) from the CCA, ECA, and/or ICA through the distal opening of the balloon catheter or other suitable arterial catheter. The aspiration may be actively initiated using a pump mechanism of a flow control device to draw fluid distal of the occlusion into the distal opening of the balloon catheter and/or other suitable arterial catheter. In some examples, the aspiration may be controlled to achieve a desired flow rate of fluid into the distal opening, but other suitable configurations are contemplated.

In some configurations, a procedural device (e.g., a stent, a stent delivery catheter, an atherectomy device, an angioplasty device, etc.) may be inserted through a working lumen of the balloon catheter or other suitable arterial catheter, where the working lumen terminates in the distal opening and receives the aspirated fluid. The procedural device may be inserted until a distal end of the procedural device is positioned at, but not necessarily protruding from, the distal opening of the working channel of the balloon catheter or other suitable arterial catheter. In some examples, while the procedure device is being inserted through the balloon catheter and/or other suitable arterial catheter, fluid may be aspirated at a nominal flow rate through the balloon catheter and/or other suitable arterial catheter (e.g., a nominal flow rate may be a flow rate expected to be lower than required to achieve full retrograde/reverse flow of fluid), but this is not required.

With the distal end of the procedural device positioned at the distal end of the working lumen that receives the retrograde/reverse flow of fluid, pump rate of the pump mechanism and/or the flow rate of aspiration may be increased to a desired pump rate or a desired flow rate selected for inducing a full retrograde/reverse fluid flow from the ECA, the ICA, and the CCA. The pump rate and/or the flow rate of fluid may be adjusted to the desired pump rate or the desired flow rate by adjusting the pump mechanism via control signals from a controller to a motor in communication with the pump mechanism. As the pump rate and/or the flow rate of fluid approaches and/or reaches the desired pump rate or the desired flow rate, sensors may sense measures (e.g., motor current, force measurements, etc.) related to a force acting on the pump mechanism to achieve the desired pump rate and/or the desired flow rate and send those measures to the controller for analysis. As the flow control device increases and reaches the desired pump rate and/or the desired flow rate, the controller of the flow control device may monitor 206 over time the received measures related to a force exerted by the pump mechanism (e.g., of a pump system) to aspirate fluid from the CCA, ECA, and the ICA.

Based on monitoring over time and analyzing the received measurements, the controller may determine or detect 208 a full or total retrograde/reverse flow of fluid in or from the ECA and the ICA. The detecting the full or total retrograde/reverse flow of fluid in or from the ECA and the ICA may include, among other analyses, identifying a period of time wherein the measure related to the force exerted by the pump system has about a first value (e.g., a period of time where the force has plateaued) while the pump is operating at the desired pump rate and/or while aspirating blood from the CCA at the desired flow rate and then, identifying the total reverse flow of blood from the ECA and the ICA when the measure related to the force exerted by the pump system increases from the first value to a second value. In some examples, the received measurements may be a motor current and a full retrograde/reverse flow of fluid from the ECA and the ICA may be detected when a slope of the motor current provided to or used by the motor is detected as increasing from a slope at a motor current plateau after a period of time (e.g., a change in slope of the motor current is a above a threshold value). In other examples, the received measurements may be a force acting on the pump mechanism and a full retrograde/reverse flow of fluid from the ECA and the ICA may be detected when a slope of the force provided to or used by the motor is detected as increasing from a slope at a force plateau after a period of time (e.g., a change in slope of the force is above a threshold value).

If a total or full retrograde/reverse flow of fluid is not detected at the desired pump rate and/or the desired flow rate, the controller may output a signal to the motor to increase the pump rate of the pump mechanism and/or the aspiration flow rate through the working lumen (e.g., by increasing a pumping rate of the pump mechanism). Then, the measures related to the force acting on the pump mechanism may be monitored for detection of the total or reverse flow of fluid from the ECA and the ICA. This process or control protocol may be repeated until the controller detects the total or reverse flow of fluid from the ECA and the ICA.

When the total or reverse flow of fluid from the ECA and the ICA is detected, the controller may run a process or control protocol for setting and maintaining the flow rate through the working channel and/or a pumping rate of the pump mechanism to maintain the total or full reverse flow of fluid from the ECA and the ICA. After detecting the total or full reverse flow of fluid from the ECA and the ICA, the process or control protocol may include reducing the pump rate of the pump mechanism and/or the flow rate of fluid through the working lumen from a first pump rate or flow rate to a second pump rate or flow rate (e.g., by reducing a pumping rate of the pumping mechanism from a first pumping rate to a second pump rate) such that a motor current or force on the pump mechanism or a measure related thereto (e.g., a slope) is just above and maintained at a current, a force, or other measure related thereto at which the total or full retrograde/reverse flow of fluid from the ECA and the ICA was detected. The second flow rate of fluid through the working channel and/or the second pumping rate of the pump mechanism at this motor current or force level on the pumping mechanism may be stored and that second flow rate and/or the second pumping rate may be used throughout the procedure to maintain the total or full reverse flow of fluid from the ECA and the ICA through a procedure with the procedural device. Such settings of the pump rate and/or the flow rate ensure the flow control device maintains a full or total retrograde/reverse flow of fluid from the EC and ICA through the working channel at all times during a procedure.

Controlling the pump mechanism to cause a desired flow rate of fluid through the working lumen while the distal end of the procedure device is at the distal opening of the working channel and monitoring over time the received measures from the sensors during such conditions, may allow for determining a flow rate of fluid in the working channel and/or a pumping rate of the pump mechanism when the total or full retrograde or reverse flow of fluid from the ECA and the ICA is detected under conditions that may represent a worst-case hydraulic diameter scenario at which a smallest amount of volume is available for fluid to enter the working channel from the CCA during a procedure. Because the flow rate and the pumping rate of the pump mechanism are determined under a worst-case scenario, the pumping rate of the pump mechanism and/or the flow rate of fluid aspirated into the arterial catheter may allow for a full retrograde/reverse flow of fluid from the ECA and the ICA under any conditions during a medical procedure distal of the occluded CCA. However, the flow control device may be configured to achieve the desired pump rate and/or the desired flow rate of fluid in the working lumen of the balloon catheter or other suitable arterial catheter when the distal end of the procedural device is not in the working lumen and/or is located at one or more locations other than at the distal end of the working lumen.

Having thus described several illustrative embodiments of the present disclosure, those of skill in the art will readily appreciate that yet other embodiments may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A reverse flow system for performing carotid artery procedures, the reverse flow system comprising:
a catheter extending from a proximal end to a distal end adapted to terminate within a common carotid artery (CCA), the catheter comprising an occlusion balloon, an inflation lumen in communication with the occlusion balloon, and a working lumen terminating at the distal end of the catheter;
a flow control device in communication with the working lumen, and
wherein the flow control device is configured to control a flow rate of fluid through the working lumen and confirm a full reverse flow of fluid in an internal carotid artery (ICA) in communication with the CCA and an external carotid artery (ECA) in communication with the CCA based on a measure related to an amount of force required for the flow control device to achieve the flow rate of the fluid through the working lumen.

2. The reverse flow system of claim 1, wherein the flow control device is configured to confirm the full reverse flow of fluid in the ICA and the ECA when the measure related to the amount of force required to achieve the flow rate of fluid through the working lumen reaches a threshold value.

3. The reverse flow system of claim 2, wherein the threshold value is a value of a slope of the measure related to the amount of force required to achieve the flow rate of fluid through the working lumen.

4. The reverse flow system of claim 1, wherein the measure related to the amount of force required to achieve the flow rate of fluid through the working lumen is a current supplied to a motor of a pump system configured to pump fluid through the working lumen.

5. The reverse flow system of claim 1, wherein the flow control device comprises a pump system configured to adjust the flow rate of fluid through the working lumen.

6. The reverse flow system of claim 5, wherein
the pump system comprises:
at least one syringe in fluid communication with the working lumen;
at least one valve, wherein a valve of the at least one valve is associated with each of the at least one syringe;
at least one motor, wherein a motor of the at least one motor is in communication with a plunger of each syringe of the at least one syringe to adjust the flow rate of fluid through the working lumen; and
at least one sensor configured to sense the measure related to the flow rate of fluid through the working lumen, wherein the measure related to the flow rate of fluid through the working lumen is a measure related to a force applied by the motor to the plunger to achieve the flow rate of fluid through the working lumen; and
the flow control device further comprises a controller configured to control operation of the at least one motor in response to the measure related to the force applied by the at least one motor to the plunger.

7. The reverse flow system of claim 5, wherein:
the pump system comprises:
an impeller in-line with the working lumen;
at least one motor, wherein the at least one motor is in communication with the impeller to adjust the flow rate of fluid through the working lumen; and
at least one sensor configured to sense the measure related to the flow rate of fluid through the working lumen, wherein the measure related to the flow rate of fluid through the working lumen is a measure related to a force applied by the at least one motor to the impeller to achieve the flow rate of fluid through the working lumen; and
the flow control device further comprises a controller configured to control operation of the at least one motor in response to the measure related to the force applied by the at least one motor to the impeller.

8. A method for establishing reverse flow in an internal carotid artery (ICA) and an external carotid artery (ECA) of a subject, the method comprising:
occluding a common carotid artery (CCA);
aspirating blood from the CCA at a flow rate through a working lumen of a catheter; and
detecting a total reverse flow of blood from the ICA and the ECA through the working lumen based on a measure related to a force required to achieve aspiring blood from the CCA through the working lumen at the flow rate.

9. The method of claim 8, wherein detecting the total reverse flow of blood comprises monitoring over time the measure related to the force exerted by a pump system to aspirate blood from the CCA through the working lumen at the flow rate.

10. The method of claim 9, wherein detecting the total reverse flow of blood comprises:
identifying a period of time wherein the measure related to the force exerted by the pump system has a first value while aspirating blood from the CCA at the flow rate; and
identifying the total reverse flow of blood from the ECA and the ICA when the measure related to the force exerted by the pump system increases from the first value to a second value while aspirating blood from the CCA at the flow rate.

11. The method of claim 10, further comprising:
in response to identifying the total reverse flow of blood, reducing the flow rate at which blood is aspirated from the CCA through the working lumen from a first flow rate to a second flow rate such that the measure related to the force exerted by the pump system remains constant above the second value.

12. The method of claim 9, wherein the measure related to the force exerted by the pump system to aspirate blood is a measure of current applied to a motor of the pump system.

13. The method of claim 9, further comprising:
in response to detecting the total reverse flow of blood, reducing the flow rate at which blood is aspirated from the CCA through the working lumen from a first flow rate to a second flow rate.

14. The method of claim 13, further comprising:
maintaining aspiration of blood from the CCA through the working lumen at the second flow rate while performing a procedure on one or more of the CCA, the ECA, and the ICA.

15. The method of claim 9, wherein the pump system comprises one or more syringes and a motor and monitoring the measure related to the force exerted by the pump system to aspirate blood from the CCA at the flow rate includes monitoring a measure related to a force required to cause a plunger of the one or more syringes to adjust positions while blood aspirates from the CCA at the flow rate.

16. The method of claim 8, further comprising:
if the total reverse flow of blood from the ICA and the ECA through the working lumen is not detected after a period of time, increasing the flow rate at which blood is aspirated from the CCA from a first flow rate to a second flow rate.

17. The method of claim 8, further comprising:
inserting a treatment device through the working lumen to a distal end of the catheter proximate the CCA, and
wherein the total reverse flow of blood from the ICA and the ECA through the working lumen is detected while the treatment device is located at the distal end of the catheter proximate the CCA.

18. A non-transitory computer readable medium storing instructions that when executed by one or more processors causes the one or more processors to:
monitor a measure related to a force exerted by a pump system to aspirate blood at a flow rate from the CCA through a working lumen of a catheter having a distal end positioned at the CCA; and
detect a total reverse flow of blood from an internal carotid artery (ICA) and an external carotid artery (ECA) through the working lumen based on the measure related to the force exerted by the pump system to aspirate blood at the flow rate from the CCA through the working lumen.

19. The non-transitory computer readable medium of claim 18, wherein when the processor is caused to detect the total reverse flow of blood, the processor is caused to:
identify a period of time wherein the measure related to the force exerted by the pump system has about a first value while aspirating blood from the CCA at the flow rate; and
identify the total reverse flow of blood from the ECA and the ICA when the measure related to the force exerted by the pump system to aspirate blood from the CCA at the flow rate increases from the first value to a second value.

20. The non-transitory computer readable medium of claim 19, wherein the instructions, when executed by the one or more processors, cause the one or more processors to:
in response to identifying the total reverse flow of blood, reduce the flow rate at which blood is aspirated from the CCA through the working lumen from a first flow rate to a second flow rate such that the measure related to the force exerted by the pump system remains constant above the second value.
